(19) United States
(12) Patent Application Publication
(10) Pub. No.: US 2009/0157099 A1
(43) Pub. Date: Jun. 18, 2009

(54) DEVICE AND METHOD FOR PLACEMENT OF TISSUE ANCHORS

(75) Inventor: VIHAR C. SURTI, Winston-Salem, NC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL, INC., WINSTON-SALEM, NC (US)

(21) Appl. No.: 12/325,735

(22) Filed: Dec. 1, 2008

Related U.S. Application Data
(60) Provisional application No. 61/014,633, filed on Dec. 18, 2007.

(51) Int. Cl. A61B 17/10; A61B 17/08; A61B 17/32

(52) U.S. Cl. 606/139; 606/151; 606/167

(57) ABSTRACT

Medical devices and methods for deploying tissue anchors for simple and reliable closure of openings in tissue are disclosed. The medical device generally includes an access sheath and a flexible puncturing device. The flexible puncturing device is sized to be slidably received by the access sheath. The flexible puncturing device has a lumen sized to receive the tissue fastener. The flexible puncturing device is operable between a first linear configuration and a second non-linear configuration. A distal end of the flexible puncturing device is laterally spaced from the access sheath in the second non-linear configuration, and preferably retroflexes to provide placement of the tissue anchors on a proximal side of the tissue.

### CROSS-REFERENCE TO RELATED APPLICATIONS

This application claims the benefit of U.S. Provisional Application Ser. No. 61/014,633 filed on Dec. 18, 2007, entitled “DEVICE AND METHOD FOR PLACEMENT OF TISSUE ANCHORS” the entire contents of which are incorporated herein by reference.

### FIELD

The present invention relates generally to medical devices and procedures for placing fasteners such as “tissue anchors” or “T-anchors”.

### BACKGROUND

Openings or perforations in the walls of internal organs and vessels may be naturally occurring, or formed intentionally or unintentionally. In order to permanently close these openings and allow the tissue to properly heal, numerous medical devices and methods have been developed employing sutures, adhesives, clips, and the like. One class of such devices is commonly referred to as visceral anchors or tissue anchors. In certain applications, the anchors are used with sutures to draw the opening closed. Tissue anchors of this type have been successfully used in closing openings, but are not without their drawbacks.

For example, when a series of anchors are placed around an opening, all of the individual sutures connected to the anchors must be collected and connected together. The Applicants have discovered that it can often be difficult to properly tension each of the individual sutures to ensure proper approximation of the tissue around the opening and complete closure thereof. This is especially critical within the gastrointestinal tract, where the travel of bacteria laden fluids outside of the tract may cause unwanted and sometimes deadly infection.

### BRIEF SUMMARY

The present invention provides medical devices and methods for deploying tissue anchors for simple and reliable closure of openings in tissue, that may be performed endoscopically and/or laparoscopically, and that offer increased versatility and control over opening closure. In one embodiment of a medical device constructed in accordance with the teachings of the present invention, an access sheath and a flexible puncturing device are provided for placing a tissue fastener through tissue. The flexible puncturing device is sized to be slidably received within an access lumen defined by the access sheath. The flexible puncturing device has a lumen sized to receive the tissue fastener. The flexible puncturing device is operable between a first linear configuration and a second non-linear configuration. A distal end of the flexible puncturing device is laterally spaced from the access sheath in the second non-linear configuration and faces generally proximally for piercing the tissue from a distal side to a proximal side of the tissue.

According to more detailed aspects of the medical device, the flexible puncturing device is retroflexed in the second configuration for engagement of the tissue. In the second configuration, the position of the distal end of the flexible puncturing device is rotated about 180 degrees relative to the position of the distal end in the first configuration, or may be rotated greater than 180 degrees. The distal end of the flexible puncturing device faces generally proximally in the second configuration. In one construction, a distal portion of the flexible puncturing device is formed of a shaped memory material. The shape memory of the flexible puncturing device may be temperature dependent, wherein the transition temperature between the first and second configurations is preferably at about body temperature. In another construction, a distal portion of the flexible puncturing device is biased to the second non-linear configuration. The flexible puncturing device is retracted substantially within the sheath in the first linear configuration, wherein the sheath overcomes the bias of the flexible puncturing device to straighten the flexible puncturing device in the first linear configuration. Preferably, a distal portion of the flexible puncturing device forms a curved shape in the second non-linear configuration, and the distal end of the flexible puncturing device is straight in the second non-linear configuration.

In one embodiment of a method for placing tissue fasteners through tissue to close an opening in the tissue, a medical device such as the one described above is employed in accordance with the teachings of the present invention. A distal end of the access sheath is positioned proximate the tissue. A flexible puncturing device is advanced through the access cannula. The flexible puncturing device retroflexes after passing beyond a distal end of the access sheath. A distal end of the flexible puncturing device is passed through the tissue from a distal side to a proximal side of the tissue. A first tissue fastener is delivered from the flexible puncturing device to the proximal side of the tissue.

According to more detailed aspects, the method may further include the step of delivering a second tissue fastener from the flexible puncturing device to the proximal side of the tissue at a second location. The first and second tissue fasteners may then be drawn closer together to close the opening, and the first and second tissue fasteners are secured together to maintain closure of the opening. The step of passing the distal end of the flexible puncture device through the tissue includes retracting the flexible puncture device to move the distal end in a proximal direction. The step of delivering the second tissue fastener includes advancing the flexible puncturing device to pass the distal end of the flexible puncturing device back through the tissue to the distal side of the tissue, rotating the flexible puncturing device, and again passing the distal end of the flexible puncturing device through the tissue from the distal side to the proximal side of the tissue. The step of delivering a second tissue fastener may be repeated with a plurality of tissue fasteners that are placed in a generally circular configuration around the opening.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a front view, partially cut away, of a medical device constructed in accordance with the teachings of the present invention;

FIG. 2 is a cross-sectional view of an access sheath forming a portion of the medical device depicted in FIG. 1;

FIG. 3 is a cross-sectional view of the medical device depicted in FIG. 1, illustrating use in a first configuration;

FIG. 4 is a cross-sectional view of the medical device depicted in FIG. 1, illustrating use in a second configuration;

FIGS. 5 and 6 are cross-sectional views of the medical device depicted in FIG. 1, illustrating steps for delivering fasteners;

FIG. 7 is a cross-sectional view showing closure of an opening in tissue using the fasteners and medical device depicted in FIG. 1; and

FIG. 8 is a plan view showing closure of an opening in tissue using the fasteners and medical device depicted in FIG. 1.

### DETAILED DESCRIPTION

The terms “proximal” and “distal” as used herein are intended to have a reference point relative to the user. Specifically, throughout the specification, the terms “distal” and “distally” shall denote a position, direction, or orientation that is generally away from the user, and the terms “proximal” and “proximally” shall denote a position, direction, or orientation that is generally towards the user.

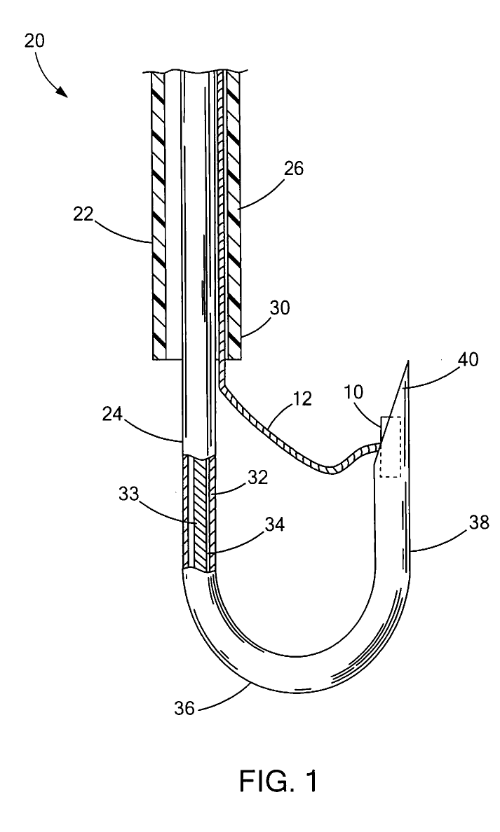

Turning now to the figures, FIG. 1 depicts a medical device 20 for placing a tissue fastener such as a T-anchor 10. T-anchors are well known in the art, exemplary T-anchors being disclosed in U.S. Pat. No. 5,123,914 and U.S. Patent Application Ser. No. 60/872,023, the disclosures of which are incorporated herein by reference in their entireties. The medical device 20 generally includes an access sheath 22 and a flexible puncturing needle 24. The access sheath 22 includes a elongate tubular body 26 defining an access lumen 28. The flexible puncturing needle 24 is slidably received within the access lumen 28. The sheath 22 and needle 24 have a length suitable for the particular application and the portion of the patient's body being accessed, such as for various endoscopic, laparoscopic and other interventional procedures. It will be recognized by those skilled in the art that many different flexible puncturing devices may be employed in place of the flexible puncturing needle 24, such as a flexible trocar or flexible electrosurgical cutting tool.

In FIG. 1, the flexible puncturing needle 24 has been shown projecting from a distal end 30 of the access sheath 22. The flexible puncturing needle 24 includes an elongate body 32 defining a needle lumen 34. The tissue anchor 10 is positioned within the needle lumen 34 for delivery through tissue, and translation of a stylet 33 relative to the needle lumen 34 deploys the tissue anchor 10, as will be described later herein. The flexible puncturing needle 24 includes a distal portion 36 that is operable between a first linear configuration (shown in FIG. 3) and a second non-linear configuration as shown in FIG. 1. A distal end 38 of the flexible puncturing needle 24 is generally linear (i.e. straight) and includes a distal tip 40 that is structured for piercing the tissue 14, as is known in the art. The distal end 38 preferably includes a slot 25 for receiving the suture 12 and preventing it from being cut by the sharpened distal tip 40.

In the second non-linear configuration (FIG. 1), the distal portion 36 takes a curved shape, and preferably a semi-annular shape as shown. Stated another way, the distal portion 36 of the flexible puncturing needle 24 retroflexes so that the distal tip 40 faces proximally. Thus, a distal tip 40 of the flexible puncturing needle 24 has been generally rotated about 180 degrees from the first configuration to the second configuration. It will be recognized that the radius of curvature in the distal portion 36, as well as the degree of bend (i.e. between at least 120 and 270 degrees) may be tailored for specific procedures and/or patients. As indicated above, the distal end 38 preferably remains straight in the second non-linear configuration for cleanly piercing the tissue 14, and thus preferably has a longitudinal length greater than or equal to a thickness of the tissue 14.

In one preferred construction, the flexible puncturing needle 24 is formed (or at least its distal portion 36 is formed) of a shape memory material such as nitinol or other similar shape memory alloys. Generally, such materials “remember” their geometry, and regain their original geometry upon heating or simply upon unloading (i.e. superelasticity). As such, the flexible puncturing needle 24 can be temperature dependent and is designed to transition between the first and second configurations at about body temperature. For example, the flexible puncturing needle 24 is introduced into the patient at a temperature below body temperature and thus in the first configuration (FIG. 3). As the flexible puncturing needle 24 is warmed to body temperature, it then assumes the second configuration shown in FIG. 1.

Suitable shape memory materials include nickel-titanium alloys (Nitinol), copper-aluminum-nickel, copper-zinc-aluminum, and iron-manganese-silicon alloys. Alternatively to using the body temperature to effect the shape memory, a higher or lower temperature fluid may be delivered to the distal end 38 of the needle 24 (e.g. via the sheath 22) at a later time to cause it to take its second position (FIG. 1) or resume its delivery configuration (FIG. 3). The distal end 38 of the needle 24 can also be formed to include a stress induced martensite (SIM) phase such that sufficient stress on the distal end 38 causes it to become more plastic and able to take the second configuration.

In another preferred construction, the flexible puncturing needle 24 is simply constructed of a resilient material such as nitinol (preferably in a superelastic state), stainless steel, other metals or alloys, or resilient plastics, and is biased towards the second configuration. In this case, the access sheath 22 is utilized to straighten the flexible puncturing needle 24 into its first linear configuration by withdrawing the distal portion 36 within the access sheath 22. In this construction, the access sheath 22 is sufficiently rigid to straighten the flexible puncturing needle 24. Accordingly, the access sheath 22 is preferably constructed of a plastic, metal or alloy that is more rigid than the flexible puncturing needle 24, or the tubular body 26 of the access sheath 22 is reinforced (e.g. with filaments or coils) or simply has a greater thickness. At the same time, the access sheath 22 retains sufficient flexibility for navigation of the body and bodily lumens, such as the gastrointestinal tract. The access sheath 22 is also preferably sufficiently flexible to be traversed through the working channel of an endoscope, whereby the endoscope can be used to navigate the bodily lumen, identify a target site, and monitor placement of the anchors 10.

It will be recognized that numerous other structures and designs of the flexible puncturing needle 24 can be utilized to achieve operability between the first and second configurations. For example, as shown in FIG. 9, a control wire 50 is operably connected to the distal end 38 of the needle 24. The distal end of the control wire 50 may be bonded to the needle 24 (e.g. adhesives, welding, soldering, etc.) or may be fixed to the needle 24 via an additional hole or slot (not shown) in the needle 24. From the distal end 38 of the needle 24, the control wire 50 extends proximally through a small hole 52 formed in the needle 24, although it could simple extend along the needle 24 and sheath 22, inside or outside their respective lumens. The hole 52 is located proximally of the attachment point between the control wire 50 and needle 24. Operation of the control wire 50, and namely a proximally directed force thereon to tension the control wire 50, facilitates transition of the puncturing needle 24 between its first and second configurations. Similarly, when the suture 12 of the tissue anchor 10 extends along the exterior of the needle 24 as shown, the suture 12 itself could be tensioned to facilitate bending of the needle 24. The access sheath 22 may also have some pre-formed curvature at its distal end 30 to facilitate the operation and placement of flexible puncturing needle 24 and the fasteners 10. It will also be recognized that the access sheath 22 and flexible puncturing needle 24 can have non-circular cross-sections.

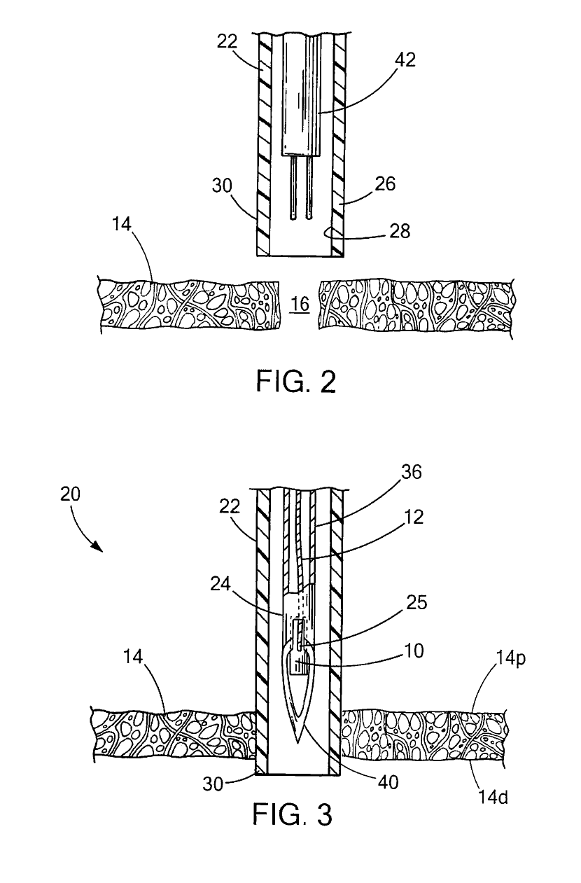

A method of placing tissue anchors 10 through tissue 14 utilizing the medical device 20 will now be described with reference to FIGS. 2-8. The medical device 20 is deployed through an opening 16 in the tissue 14. The opening 16 may be naturally occurring, or may be intentionally or unintentionally formed. For example, the opening 16 may be intentionally formed utilizing the access sheath 22 of the medical device 20 and an electrosurgical cutting tool 42 (FIG. 2). The electrosurgical cutting tool 42 is generally advanced through the access lumen 28 and positioned to project from a distal end 30 of the access sheath 22. The cutting tool 42 is operated to form the opening 16 in the tissue 14. It will be recognized by those skilled in the art that many types of elongate cutting devices may be employed, and likewise an endoscope or other visualization tools may be employed in conjunction with the cutting device to select an access site and form the opening 16 in the tissue 14.

Turning now to FIG. 3, the access sheath 22 is inserted into the opening 16. If needed, a dilator (not shown) may be employed to enlarge the opening 16. The dilator, such as a balloon catheter, can also be deployed through the access lumen 28 of the access sheath 22. As shown in the figure, the medical device 20, and in particular the flexible puncturing needle 24, is in its first linear configuration. Preferably, the distal end 30 of the access sheath 22 is advanced well beyond a distal side 14d of the tissue 14, thereby ensuring sufficient clearance from the tissue 14 for the flexible puncturing needle 24 to transition into its second non-linear configuration.

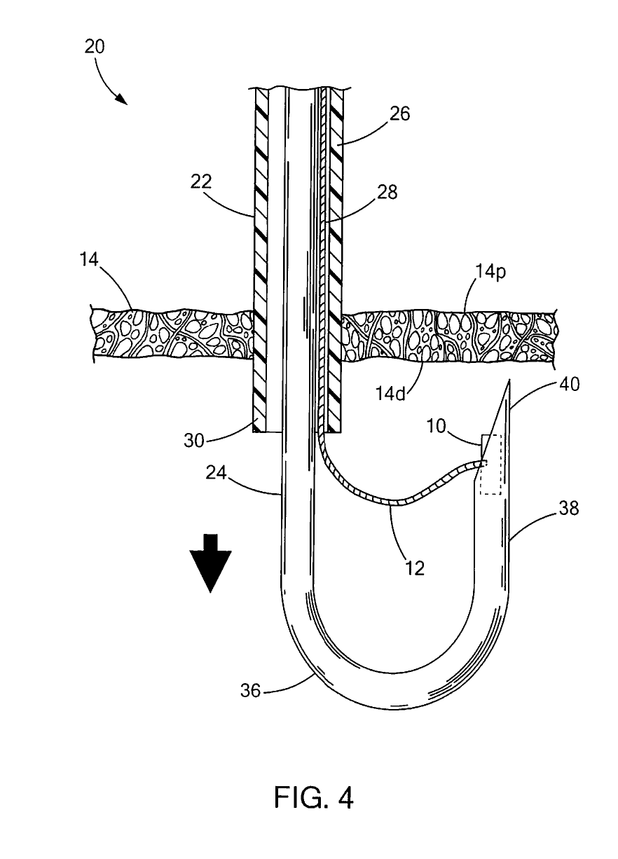

As shown in FIG. 4, the flexible puncturing needle 24 is advanced through the access lumen 28 of the access cannula 22, as shown by the arrow in FIG. 4. Due to the construction of the flexible puncturing needle 24, the flexible puncturing needle 24 retroflexes after passing beyond the distal end 30 of the access sheath 22. As previously noted, the distal portion 36 takes a curved shape while the distal end 38 and distal tip 40 remain substantially straight. Through this construction, the flexible puncturing needle 24 may be passed straight through the tissue 14, thereby avoiding unfavorable angles for deployment of the anchors 10. As such, the distal end 38 preferably has a longitudinal length greater than or equal to a thickness of the tissue 14.

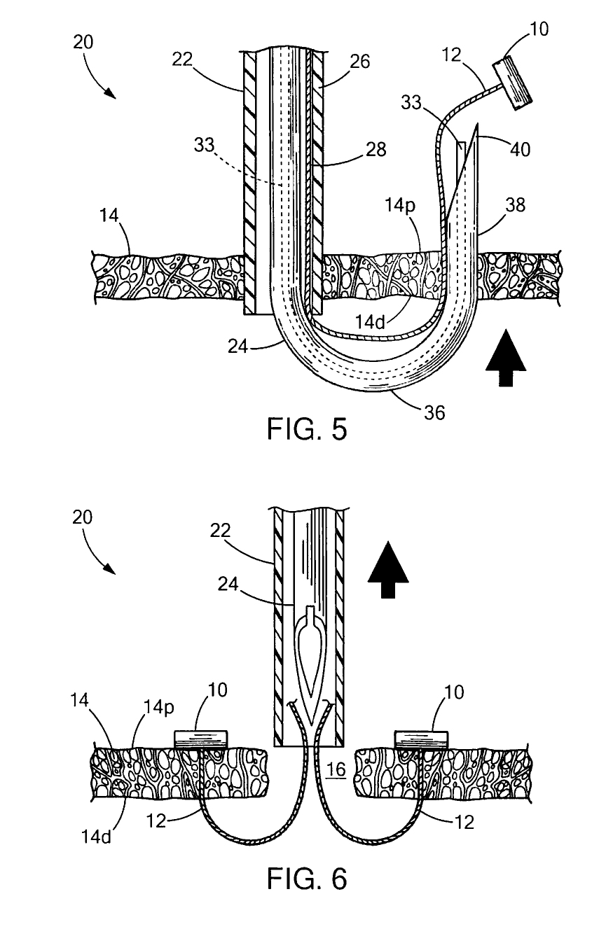

When the flexible puncturing needle 24 has attained its second non-linear configuration, the needle 24 is retracted (i.e. translated proximally) as indicated by the arrow in FIG. 5. Through retraction of the flexible puncturing needle 24, the distal end 38 and distal tip 40 of the flexible puncturing needle 24 are passed through the tissue 14 from the distal side 14d to the proximal side 14p of the tissue 14. As also shown in FIG. 5, the access sheath 22 may be retracted a small amount to ensure the flexible puncturing needle 24 has sufficient clearance to pass completely through the tissue 14. The flexible puncturing needle 24 is directed generally perpendicular to the tissue 14, resulting in the anchors 10 being placed through the tissue 14 at favorable angles. The high level of control provided by the medical device 20 thus insures that a minimal number of anchors 10 can be used. The tissue anchor 10 may then be advanced from the flexible puncturing needle 24 and its needle lumen 34 to the proximal side 14p of the tissue 14. The stylet 33 is translated distally relative to the needle 24, thereby deploying the anchor 10 as shown. The anchor 10 remains connected to the suture 12, which has been shown as passing through the access lumen 28 of the access sheath 22 to a location outside of the body. It will be recognized by those skilled in the art that the suture 12, rather than passing through the access lumen 28, may pass through the needle lumen 34 or extend along the outer periphery of the access sheath 22.

The flexible puncturing needle 24 may then be advanced distally to withdraw the distal end 38 and distal tip 40 from the tissue 14, to a location on the distal side 14d of the tissue 14 (such as is shown in FIG. 4). Once withdrawn from the tissue 14, the flexible puncturing needle 24 may be retracted, reloaded with another tissue anchor 10, and returned to the second non-linear configuration. It will be recognized that in anchor delivery systems where a series of tissue anchors may be delivered through the needle lumen 34 of the flexible puncturing needle 24, the needle 24 need not be completely withdrawn from the access sheath 22, but rather may be preloaded or reloaded from the proximal end of the needle 24 as needed.

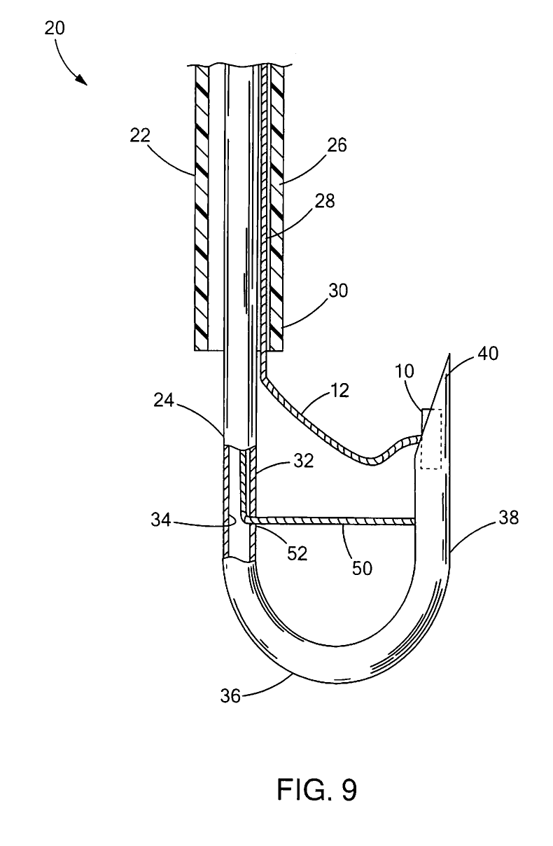

With a second tissue anchor 10 loaded, the needle 24 is rotated (i.e. twisted or turned) to select another site in the tissue 14 around the periphery of the opening 16, for placement of a second tissue anchor 10. In this manner, the aforementioned steps may be repeated to place a plurality of anchors 10 circumferentially about the opening 16. As shown in the plan view of FIG. 8, utilization of the medical device 20 and the described procedure may be repeated to place a plurality of tissue anchors 10 around the opening 16 along a generally circular path. While four tissue anchors 10 have been depicted, it will be recognized that any number of anchors 10 may be used depending upon the particular situation. To assist with placement and location of the tissue anchors 10, indicia may be provided on the proximal ends of the flexible puncturing needle 24 and/or the access sheath 22. Likewise, the procedure may be performed under fluoroscopy, using ultrasound guidance, or using other now known or future developed monitoring techniques.

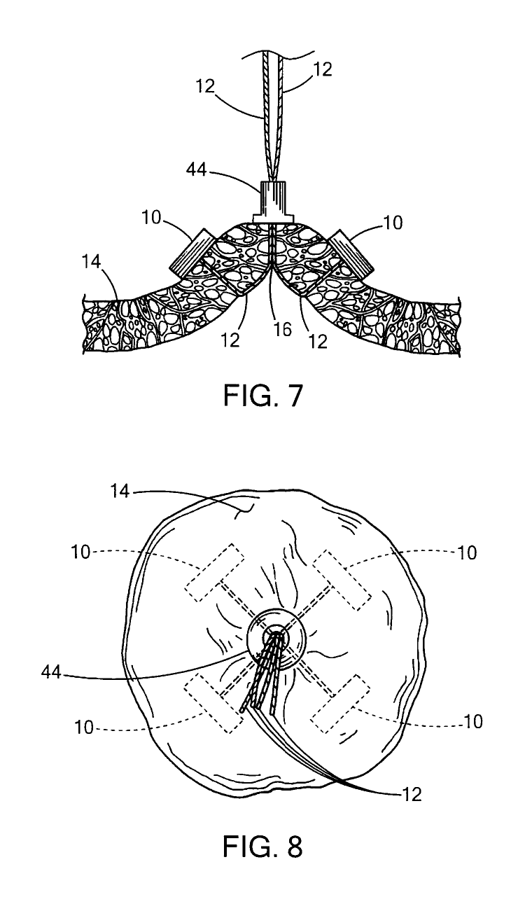

As shown in FIG. 6, after placement of a plurality of tissue anchors 10, the medical device 20, namely the access sheath 22 and flexible puncturing needle 24, may be withdrawn as indicated by the arrow in the figure. The plurality of tissue anchors 10 remain on the proximal side 14p of the tissue 14, while the corresponding sutures 12 extend through the tissue 14, along the distal side 14d, and then extend proximally through the opening 16 in the tissue 14. As shown in FIG. 7 when the medical device 20 (or at least the flexible puncturing needle 24) has been withdrawn, a suture lock 44 may be utilized to fix the relative positions of the sutures 12 and close the opening 16. The sutures 12 are placed in tension to draw the anchors 10 closer together and close the perforation 16. Exemplary suture locks are disclosed in U.S. Patent Application Nos. 60/941,086 filed May 31, 2007 and 60/956,575 filed Aug. 17, 2007, the disclosures of which are incorporated herein by reference in their entireties. Tension on the sutures 12 may be adjusted prior to fixing them with the suture lock 44 in order to completely close the opening 16. Notably, by placing the anchors 10 on the proximal side 14p of the tissue 14, the anchors 10 do not fight the collapsing of the opening 16, but rather promote the complete closure of the opening 16. That is, the tension placed on the sutures 12, with the sutures 12 extending along the distal side 14d and directly through the opening 16 to the proximal side 14p of the tissue 14, results in the anchor 10 and sutures 12 inducing an inwardly directed compressive force on the opening 16 to facilitate complete closure thereof. As such, reliable and complete closure of the opening 16 may be obtained to promote healing and prevent leaking of fluids through opening 16.

While the medical procedure has been described as positioning the access sheath 22 within the opening 16 prior to advancement of the flexible puncturing needle 24, it will be recognized by those skilled in the art that the flexible puncture needle 24 itself may be used to form the opening 16. With the access sheath 22 positioned proximate the tissue 14, the flexible puncturing needle 24 is advanced through the access lumen 28 and beyond the distal end 30 to puncture the tissue and form the opening 16. As such, the flexible puncturing needle 24 will again retroflex upon further advancement of the needle 24, and the needle 24 may then be passed through the tissue 14 from the distal side 14d to the proximal side 14p. It will also be recognized by those skilled in the art that the distal end 38 of the flexible puncturing needle 24 need not be straight, but rather can form part of the curved distal portion 36 of the flexible puncturing needle 24. In fact, this construction of the flexible puncturing needle 24 is preferred when the access sheath 22 is not intended to be advanced into or through the opening 16 in the tissue 14. Finally, while the needle 24 has been described as being first distally advanced (FIG. 4) and then proximally retracted (FIG. 5) to pierce the tissue 14, the curvature of the needle 24 at its distal end 36 may be structured to pierce the tissue 14 through only the advancement of the needle 24, such as by having a curvature turning greater than 180 degrees.

Utilizing the above-described devices and methods, simple and reliable closure of openings in tissue is provided. By placing the anchors 10 on the proximal side 14p of the tissue 14, better closure of openings or perforations is provided. Although applicant is not to be limited to any particular theory, it is believe that the tension on the sutures 12, spanning from the anchors 10 (located around the periphery of the opening 16 and on the proximal side 14p), along the sutures 12 on the distal side 14d, to the suture lock 44 (located generally at the center of the opening 16 and on the proximal side 14p), facilitates improved closure of the opening 16. The devices are simple to operate, and the methods may be performed endoscopically and/or laparoscopically. Finally, the devices and methods offer increased versatility and control over perforation closure, as any number of tissue anchors 10 may be employed and are easily spaced in a circumferential configuration around the opening 16.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A medical device for placing a tissue fastener through tissue, the medical device comprising:
an access sheath defining an access lumen; and
a flexible puncturing device sized to be slidably received by the access lumen of the access sheath, the flexible puncturing device defining a lumen sized to receive the tissue fastener, the flexible puncturing device operable between a first linear configuration and a second non-linear configuration, a distal end of the flexible puncturing device being laterally spaced from the access sheath in the second non-linear configuration and facing generally proximally to pierce the tissue from a distal side to a proximal side of the tissue.

2. The medical device of claim 1, wherein the flexible puncturing device is retroflexed in the second configuration for engagement of the tissue.

3. The medical device of claim 1, wherein in the second configuration, the position of the distal end of the flexible puncturing device is rotated about 180 degrees relative the position of the distal end in the first configuration.

4. The medical device of claim 1, wherein in the second configuration, the position of the distal end of the flexible puncturing device is rotated greater than 120 degrees relative the position of the distal end in the first configuration.

5. The medical device of claim 1, wherein a distal portion the flexible puncturing device is formed of a shape memory material that is temperature dependent, and wherein the transition temperature between the first and second configurations is at about body temperature.

6. The medical device of claim 1, wherein a distal portion of the flexible puncturing device is biased to the second non-linear configuration.

7. The medical device of claim 6, wherein the flexible puncturing device is retracted substantially within the access sheath in the first linear configuration.

8. The medical device of claim 7, wherein the sheath overcomes the bias of the flexible puncturing device to straighten the flexible puncturing device in the first linear configuration.

9. The medical device of claim 1, wherein a distal portion of the flexible puncturing device forms a curved shape in the second non-linear configuration, and wherein the distal end of the flexible puncturing device is straight in the second non-linear configuration.

10. The medical device of claim 9, wherein the distal portion of the flexible puncturing device is biased to the curved shape in the second non-linear configuration, and wherein the distal end is not biased.

11. The medical device of claim 9, wherein the distal portion the flexible puncturing device is formed of a shape memory material that transitions between a generally straight shape and the curved shape, and wherein the distal end is constructed to not transition to a curved shape.

12. The medical device of claim 9, wherein the distal end has a longitudinal length greater than or equal to a thickness of the tissue.

13. The medical device of claim 1, further comprising a control wire fixed to a distal portion of the flexible puncturing device and extending proximally, and wherein proximal tension on the control wire facilitates operation of the flexible puncturing device between its first and second configurations.

14. The medical device of claim 13, wherein the flexible puncturing device includes a hole formed therein at a location proximal to the location where the control wire is fixed to the distal portion of the flexible puncturing device, and wherein the control wire extends from the exterior of the distal portion, through the hole, and through the lumen of the flexible puncturing device.

15. A method for placing tissue fasteners through tissue to close an opening in the tissue, the method comprising the steps of:
positioning an access sheath proximate the tissue, the access sheath defining a lumen;
advancing a flexible puncturing device through the access lumen of the access sheath,
retroflexing the flexible puncturing device such that a distal end of the flexible puncturing device faces proximally;
passing a distal end of the flexible puncturing device through the tissue from a distal side to a proximal side of the tissue; and
delivering a first tissue fastener from the flexible puncturing device to the proximal side of the tissue.

16. The method of claim 15, further comprising the step of delivering a second tissue fastener from the flexible puncturing device to the proximal side of the tissue at a second location.

17. The method of claim 15, wherein the step of passing a distal end of the flexible puncturing device through the tissue includes retracting the flexible puncture device such that the distal end moves in a proximal direction.

18. The method of claim 16, wherein the step of delivering the second tissue fastener includes,
advancing the flexible puncturing device to pass the distal end of the flexible puncturing device back through the tissue from the proximal side to the distal side of the tissue,
rotating the flexible puncturing device, and
passing the distal end of the flexible puncturing device through the tissue from the distal side to the proximal side of the tissue.

19. The method of claim 18, wherein the step of delivering a second tissue fastener is repeated with a plurality of tissue fasteners that are placed in a generally circular configuration around the opening.

20. The method of claim 15, wherein the access sheath is inserted through the opening.
